(19) United States
(12) Patent Application Publication
(10) Pub. No.: US 2023/0414368 A1
(43) Pub. Date: Dec. 28, 2023

(54) OSTEOINDUCTIVE IMPLANTS AND RELATED MANUFACTURING METHODS

(71) Applicant: Spectrum Spine IP Holdings, LLC, Inlet Beach, FL (US)

(72) Inventors: James C. Robinson, Inlet Beach, FL (US); Timothy Ganey, Tampa, FL (US)

(73) Assignee: Spectrum Spine IP Holdings, LLC, Inlet Beach, FL (US)

(21) Appl. No.: 18/340,014

(22) Filed: Jun. 22, 2023

Related U.S. Application Data
(63) Continuation-in-part of application No. 17/942,420, filed on Sep. 12, 2022, now Pat. No. 11,771,528.
(60) Provisional application No. 63/354,758, filed on Jun. 23, 2022.
(60) Provisional application No. 63/506,050, filed on Jun. 2, 2023.

(51) Int. Cl. A61F 2/30; A61L 27/06

(52) U.S. Cl. CPC A61F 2/30767; A61L 27/06; A61F 2310/00023; A61L 2400/18; A61F 2002/30925; A61F 2002/3092

(57) ABSTRACT

An implant device configured to be at least partially in contact with bone on implantation and having an improved osteoinductive feature to enhance new bone formation. The implant device has one or more bone growth surfaces extending from a structurally solid feature of the implant device. The one or more bone growth surfaces are configured to mimic adult trabecular bone by having recesses and prominences. The recesses extend 10 to 500 microns in depth and may have an increasing inclination from the surface extending inwardly and not parallel to opposing or adjacent walls. The one or more bone growth surfaces form a random and/or non-random network.

### FIELD OF THE INVENTION

Osteoinductive implants having surfaces modified for enhanced bone growth formation. Methods for manufacturing the implant devices provide surface features mimicking those of trabecular bone.

### BRIEF DESCRIPTION OF SEVERAL VIEWS OF THE DRAWINGS

FIG. 1A is a photograph providing a perspective view of an osteoinductive implant device with surfaces that have been etched and treated.

FIG. 1B is an enlarged perspective view of the osteoinductive implant device depicted in FIG. 1A showing the surfaces that have been etched and treated.

FIG. 1C is a plan view of surfaces of a device, after magnification, that have been laser etched and laser treated.

FIG. 1D is a scanning electron micrograph (SEM) image of a laser-treated surface.

FIG. 1E is an SEM image of the laser-treated surface shown in FIG. 1D after further magnification.

FIG. 1F is an SEM image of a laser-treated surface under 200,000× magnification with a scale of 200 nm.

FIG. 2 is a perspective view of an exemplary laser.

FIG. 3 is a perspective view of an exemplary 3D printer.

FIG. 4A depicts a perspective view of a test disk.

FIG. 4B is a planar image of a test disk with an etched area and a cutting line 4C-4C between points A and B.

FIG. 4C is an image depicting the measured depths of the recesses and surfaces along cutting line 4C-4C as measured by a profilometer.

FIG. 4D is a planar image of an enlarged section of the test disk shown in FIG. 4A with a cutting line 4E-4E between and with 12 points along the cutting line 4E-4E.

FIG. 4E depicts profile topographic mapping of the depths of the recesses and surfaces at the 12 points identified in FIG. 4D.

FIG. 4F depicts the same enlarged section depicted in FIG. 4D with a few select recesses identified as recesses #1-#6 and with horizontal cutting line 4G-4G and a vertical cutting line 4H-4H.

FIG. 4G is an image depicting the measured depths of the recesses and surfaces along cutting line 4G-4G as measured by a profilometer.

FIG. 4H is an image depicting the measured depths of the recesses and surfaces along cutting line 4H-4H as measured by a profilometer.

FIG. 5 is a photograph of seven disks that were samples subjected to comparative analysis when viewed via scanning electron microscopy (SEM) in which the samples have differing recess trench depths via laser etching, use of surface texturing, and a control.

FIG. 6A is a microscopic image of Sample #1, which has etched recesses having a depth of 100 microns, at a level of 20× magnification of a section of Sample #1 having a length of 2000 μm.

FIG. 6B is a microscopic image of a section of Sample #1 in FIG. 6A with a length of 500 μm and further magnified to a level of 120× magnification.

FIG. 6C is a microscopic image of a section of Sample #1 in FIG. 6A with a length of 500 μm and further magnified to a level of 200× magnification.

FIG. 7A is a microscopic image of Sample #2, which has etched recesses having a depth of 200 microns, at a level of 20× magnification of a section of Sample #1 having a length of 2000 μm.

FIG. 7B is a microscopic image of a section of Sample #2 in FIG. 7A with a length of 500 μm and further magnified to a level of 120× magnification.

FIG. 7C is a microscopic image of a section of Sample #2 in FIG. 7A with a length of 200 μm and further magnified to a level of 200× magnification.

FIG. 8A is a microscopic image of Sample #3, which is another disk that like Sample #1 with etched recesses having a depth of 100 microns, at a level of 20× magnification of a section of Sample #3 having a length of 2000 μm.

FIG. 8B is a microscopic image of a section of Sample #3 in FIG. 8A with a length of 500 μm and further magnified to a level of 120× magnification.

FIG. 8C is a microscopic image of a section of Sample #3 in FIG. 8A with a length of 200 μm and further magnified to a level of 200× magnification.

FIG. 9A is a microscopic image of Sample #4, which is another disk like Sample #2 with etched recesses having a depth of 200 microns, at a level of 20× magnification of a section of Sample #1 having a length of 2000 μm.

FIG. 9B is a microscopic image of a section of Sample #4 in FIG. 9A with a length of 500 μm and further magnified to a level of 120× magnification.

FIG. 9C is a microscopic image of a section of Sample #4 in FIG. 9A with a length of 200 μm and further magnified to a level of 200× magnification.

FIG. 10A is a microscopic image of Sample #5, which was treated with a laser that blasted the surfaces of the disk to yield nano-textured surfaces with nano-sized features. FIG. 10A is a section of the disk, at a level of 20× magnification of a section of Sample #5 having a length of 2000 μm.

FIG. 10B is a microscopic image of a section of Sample #5 in FIG. 10A with a length of 500 μm and further magnified to a level of 120× magnification.

FIG. 10C is a microscopic image of a section of Sample #5 in FIG. 10A with a length of 200 μm and further magnified to a level of 200× magnification.

FIG. 11A is a microscopic image of Sample #6, which is another disk like Sample #5 treated with a laser that blasted the surfaces of the disk to yield nano-textured surfaces with nano-sized features. FIG. 11A is a section of the disk, at a level of 20× magnification of a section of Sample #6 having a length of 2000 μm.

FIG. 11B is a microscopic image of a section of Sample #6 in FIG. 11A with a length of 500 μm and further magnified to a level of 120× magnification.

FIG. 11C is a microscopic image of a section of Sample #6 in FIG. 11A with a length of 200 μm and further magnified to a level of 200× magnification.

FIG. 11D is a microscopic image of a section of Sample #6 in FIG. 11A with a length of 200 μm and further magnified to a level of 400× magnification.

FIG. 12 is a microscopic image of a section of Sample #7 with a length of 500 μm and magnified to a level of 120× magnification.

FIG. 13 is a photograph focused on the surface of a device showing the growth of cellular bone tissue around trenches and on the treated surfaces.

FIG. 14 is an SEM image showing the bone growth on a laser treated device.

FIG. 15 is a schematic representation of a recess formed by laser etching a solid feature of an implant device to show some asymmetry when forming a recess based on trabecular bone 3D morphology and geometry.

FIG. 16A is an exemplary embodiment of a device such as a spinal implant fusion device formed at least in part by an additive process.

FIG. 16B is a cross-sectional view of the implant device of FIG. 16A taken along cutting line 16B-16B of FIG. 16A.

FIG. 16C is an enlarged view of the surface of the device in FIG. 16A showing laser etched nano channels.

FIG. 17 is an enlarged view of a portion of an undulating exterior surface with protruding and depressed features at the surface mimicking trabecular bone.

FIG. 18 is a simplified schematic outline of a portion of an exterior surface showing projections and channels or troughs of an alternative embodiment.

FIG. 19A is a simplified schematic drawing showing the making of laser etched channels using a moving laser machine.

FIG. 19B is a simplified schematic drawing showing the making of laser etched channels using a fixed laser machine with the implant being moved.

### DETAILED DESCRIPTION

Implant devices are disclosed that are configured to be at least partially in contact with bone on implantation and having an osteoinductive feature to enhance new bone formation. The related methods for manufacturing the devices are also disclosed.

Implant devices that are embedded into bone are ideally secured to the bone by new bone growth formations that extend onto and into the bone growth surfaces or adapt bone formation to that attaches to the implant device. Appreciation for the reciprocal responses between biologic tissues and material devices has focused on recreating specific environmental, with surface features and conditions that might best simulate a mechanistic approach to the mechanobiological response of cells.

Such implant devices include spinal fusion cages, bone screws and other bone fasteners, plates used in bone fracture repairs, knee and hip repair devices, pedicle screws, cervical plates, non-spinal orthopedic implants, dental implants including abutments that when implanted into the jawbone at the gums and other devices used to stabilize bones for bone repair procedures exert physical effects that align and/or amplify normal biologic process. Essentially, any bone-interfacing device that benefits from bone growth into and/or around the surface of the implant may be modified to create the bone-growth surfaces on an existing implant device. All of these devices are firmly secured to the bone by new bone growth that extends to and surrounds the bone growth surfaces of the implant device.

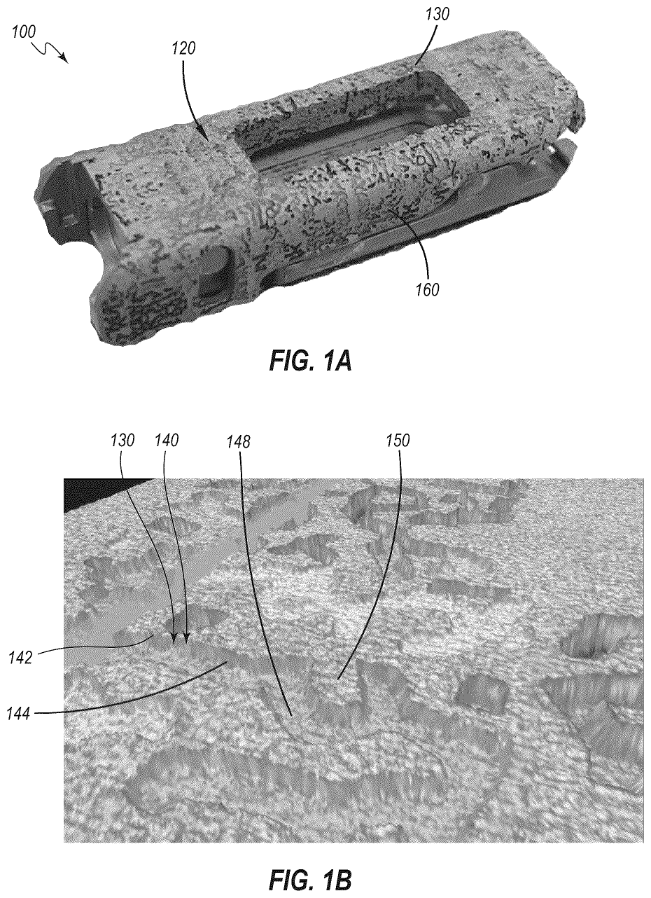

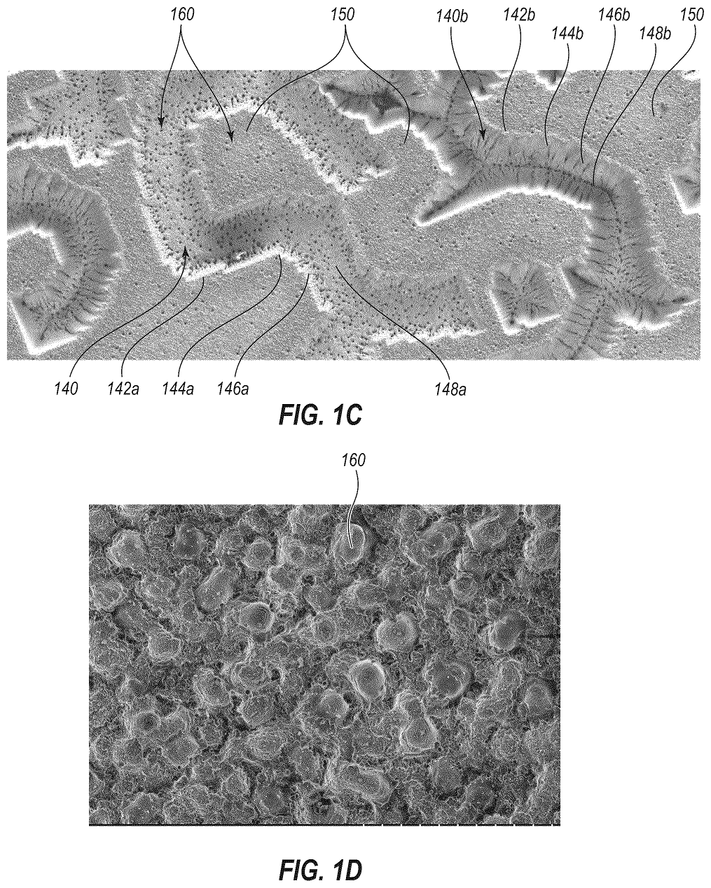

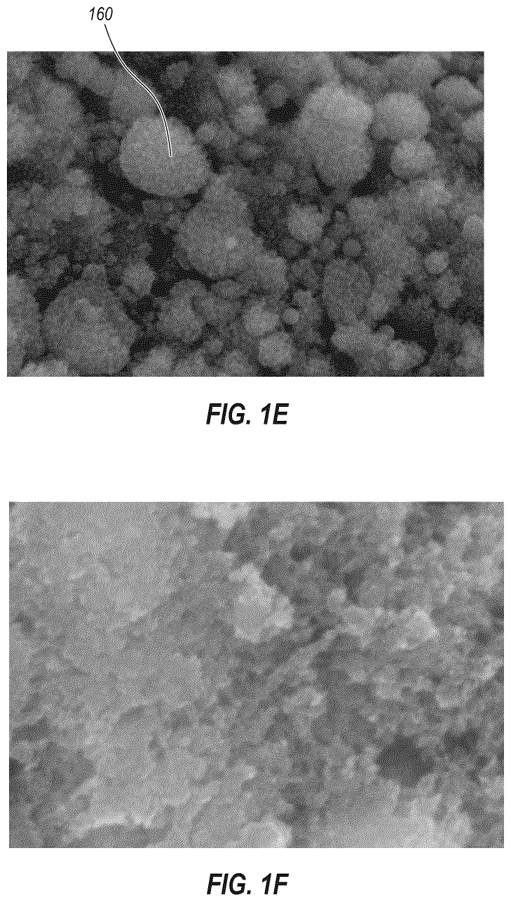

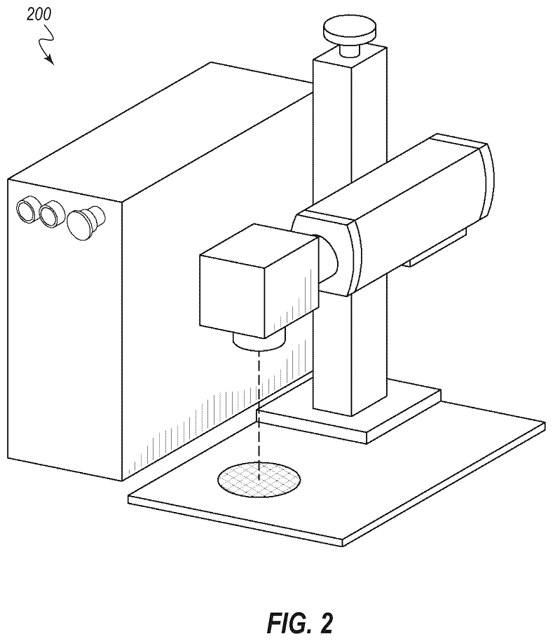

By way of example, one such device is a spinal implant device as shown in FIG. 1A at 100. Surgical implantation of interbody cages is typically used to provide support along the spinal column in cases where a portion of the patient's intervertebral anatomy has become weakened, diseased or destroyed, or compromised in surgical intervention guided to achieve anatomical stability. Such support systems are also commonly used following a discectomy, where an intervertebral disc is surgically removed. Most commonly, existing support systems typically operate by inhibiting normal movement between the adjacent vertebrae, thereby stabilizing these vertebrae at fixed positions relative to one another, with the mechanical body of the supporting structure providing the needed support along the patient's spinal column. Such supporting systems are typically made of stainless steel, titanium, titanium alloy, polymer (e.g., an organic polymer thermoplastic such as polyether ether ketone (PEEK)), polyether ketone ketone (PEKK), carbon fiber, ceramic, combinations such as metal-ceramic (Cermet), or combinations of ceramic and thermoplastic designed to permanently remain within the patient's body. Any bone-interfacing device formed from these materials may be modified to have bone-growth surfaces.

It is beneficial, in addition to fixation, to try to stimulate bone growth between the adjacent vertebrae. To do so, spine surgeons often use bone graft material in addition to fixation devices. Bone graft does not heal or fuse the spine immediately; instead, bone graft provides a foundation, scaffold, or impetus for the patient's body to grow new bone. Not intended to be an impediment to motion, bone graft serves an inductive intention to stimulate new bone production. When new bone grows and solidifies, fusion occurs. Although instrumentation (e.g., screws, rods) is often used for initial stabilization (post-operative), it is the healing of bone that welds vertebrae together to create long-term stability. There are two general types of bone grafts: actual bone and bone graft substitutes. Genuine bone can come from the patient (autograft) or from a donor bone (allograft). Also used in these types of surgery are bone substitute, osteoinductive agents, stem cell products, bone morphogeneic proteins, and bone cement. The bone implant devices, as disclosed herein, have features that facilitate new bone growth to achieve hastened attachment and fusion to the patient's bone.

The material the implant device is made of may be any suitable implant material as described above for a supporting system. Appropriate materials include metal, cermet, plastic or bone in which the benefits of enhanced osteoinductivity can be achieved while maintaining appropriate anatomical space.

The implant device has one or more bone growth surfaces extending from a structurally solid feature of the implant device because the bone growth surfaces are not formed as a coating. The one or more bone-growth surfaces may be configured to mimic the structure of adult trabecular bone with bone tissue organized into a network of interconnected walls, rods, plates and arcs called trabeculae.

The bone-growth surfaces include primary surface features or structures and in some embodiments may also include secondary surface structures that are the treated surfaces of the primary surface structures. FIG. 1A shows surfaces 120 including primary surface structures 130 and secondary surface structures 160. In one embodiment, the primary surface features and the secondary surface structures are formed a laser such as the laser system depicted at 200 in FIG. 2. The spinal implant device depicted in FIG. 1A has a length of 35 mm yet it is clearly textured such that the surface recesses and prominences can be easily seen by eye without magnification.

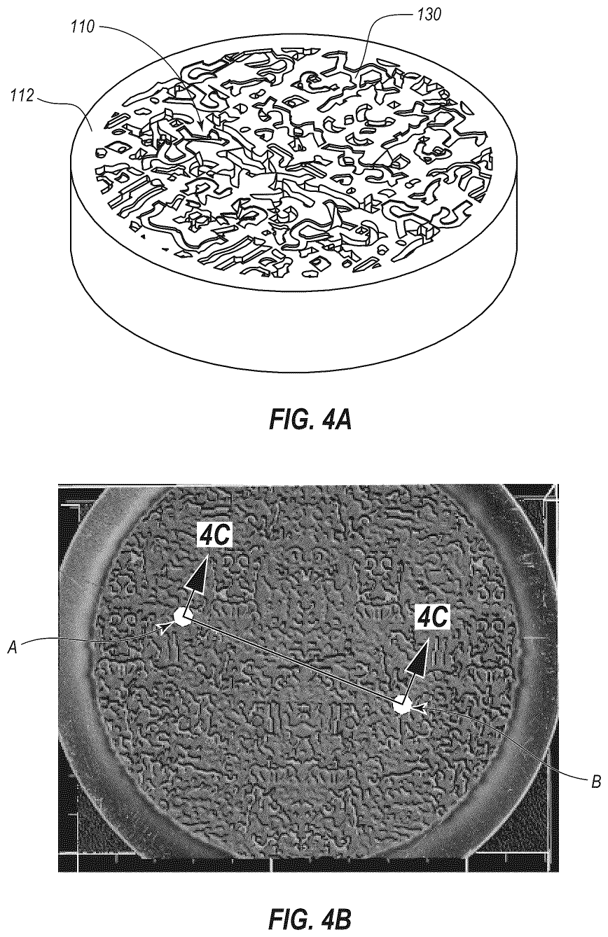

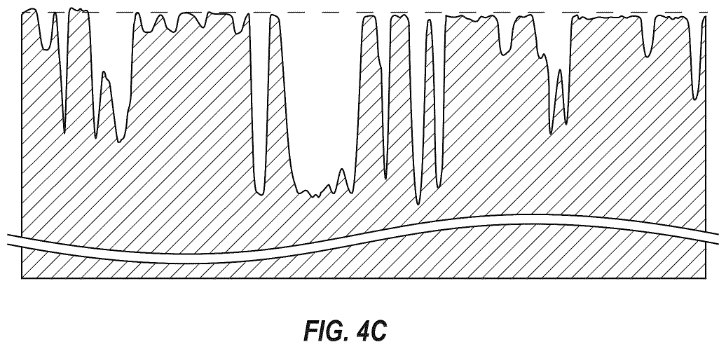

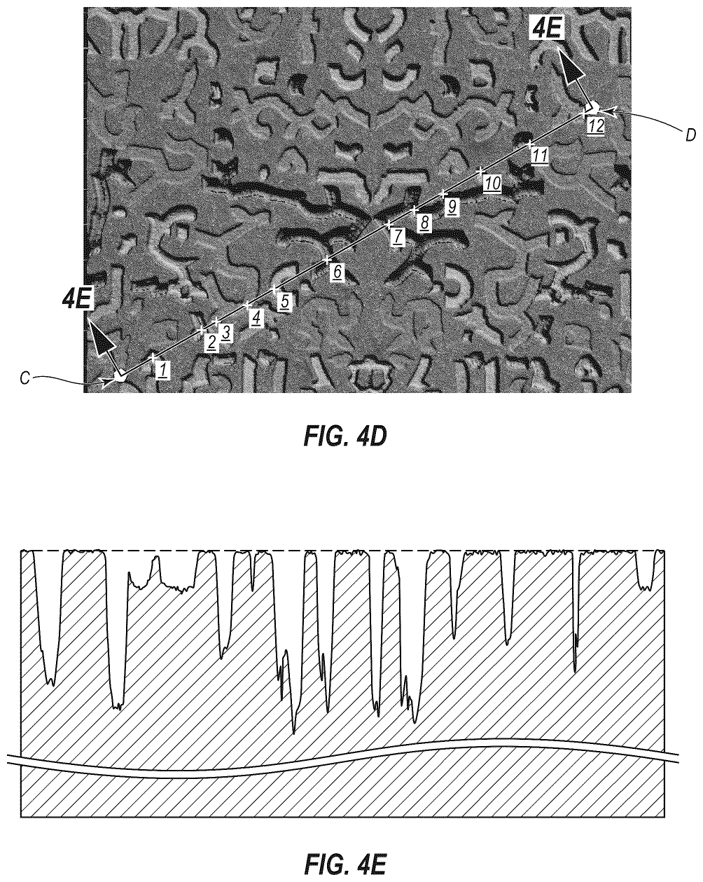

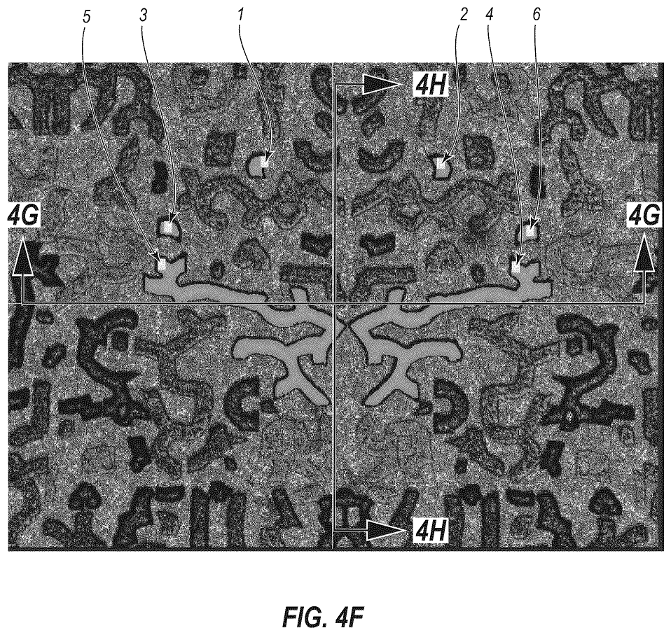

FIG. 1B is a magnified view of a section of the spinal implant device 100 shown in FIG. 1A and depicts primary surface structures 130 and secondary surface structures 160. The primary surface structures are formed in a non-random, engineered pattern, which on a macroscale may look random but algorithmically it is not random. The primary surface structures are large enough that they can be seen with the naked eye because they may be measured on a macro or microscale such as the pattern depicted in FIG. 4A. FIG. 4A depicts a test disk 110 having a diameter of 9 mm, an unetched border 112 that is 0.5 mm wide, and primary surface structures 130, which comprises an etched area having a diameter of 8 mm.

The secondary surface structures, as shown at 160 in FIG. 1B, are random in comparison to the pattern of the primary surface structures, as shown at 130 in FIG. 1B. The primary surface structures have dimensions that are measured in microns or millimeters while the secondary features have dimensions that are significantly smaller as they may be measured in nanometers such as 200 nanometers. Based on the foregoing, the implant devices have primary surface structures that are ordered or patterned and measured at a macro or microscale while at a nanometer scale or nanoscale, the secondary surface structures are less ordered or patterned than the primary surface structures, such that the nanoscale secondary surface structures appear relatively random or at least partially random. Stated more succinctly, when viewed from a macroscale or microscale to nanoscale, the surfaces trend from patterned to random.

The secondary surface structures individually create an improved osteoinductive effect at the surface of the implant device 100. This means that the formation of new bone once implanted into the patient can be accelerated and the network 18 of recesses and prominences with secondary surface structures provide features that help assist in providing attachment locations for the new bone formation. This continuous and progressive architecture with z-vector variation in addition to the macro-surface geometry is an important aspect of the embodiments disclosed herein.

The primary surface structures may be produced by a subtractive laser process in which surfaces of a device are laser etched via a laser system such as the laser 212 depicted in FIG. 2 and an associated laser computer system. Etching or ablation of the solid increases the surface area of the device. When laser etching a device, the resulting surface features or structural recesses may have depth, width, and length dimensions generally under about 1 mm (1,000 μm), and in some cases only a few microns in size, with a high degree of repeatability and without causing significant structural damage to the surrounding material. The subtractive process may also be a machining process.

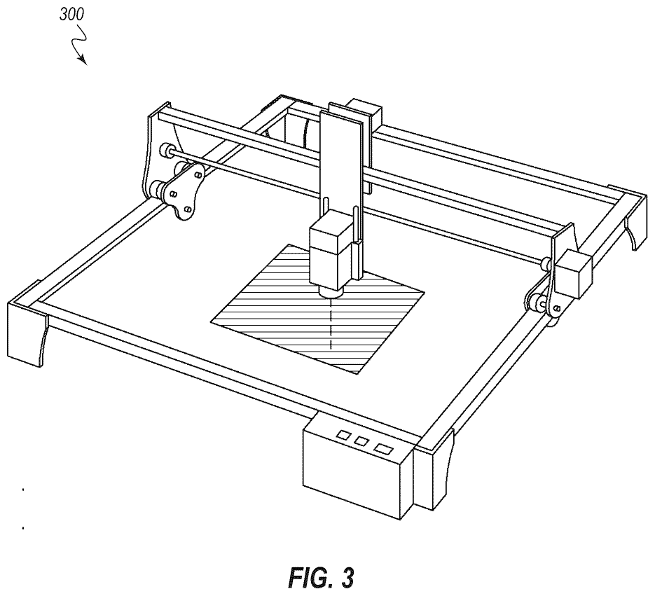

Alternatively, the primary surface structures of the device may be manufactured by an additive process using 3D printing. 3D printing is the construction of a three-dimensional object from a computer aided design (CAD) model or a digital 3D model in which material is deposited, joined or solidified under computer control to create a three-dimensional object, with material being added together, typically layer by layer via a 3D printer as shown at 300 in FIG. 3. Whether the implant device and the primary surface structures are formed by an additive process or a subtractive process, the surface may be further modified or treated by a subtractive process to nano-sculpt the surface of the device to yield the secondary surface structures or features. Additional information about manufacturing devices via an additive process using 3D printing is disclosed below under the heading “Additive Structures.” Also, information about 3D printing is also disclosed in Application No. 63/354,748, which was filed on Jun. 23, 2022 and application Ser. No. 17/942,420, which was filed on Sep. 12, 2022. Application No. 63/354,748 and application Ser. No. 17/942,420, are incorporated herein in their entirety.

By way of example, the engineered pattern may be a custom pattern developed with a CAD program based on a three-dimensional image of a section of structures at the surface of an adult trabecular bone sample, such as a 2×2 cm section that is then used to trace the surfaces and is transformed into instructions for a computer-directed laser system. The image of the section may be used to form repetitive patterns or to form identical patterns on identical devices at the same locations of the identical devices. The pattern or geometrical configuration of the primary surface structures may seem random to a casual observer at first glance but upon more careful inspection or by comparing identical devices, it should be apparent that the pattern is engineered. The initial impression may be somewhat like first looking at a quick response (QR) code or a digitized pattern for camouflage and then realizing that the pattern is highly engineered after more careful study.

In addition to generating mimetic patterns such as those based on trabecular bone, the engineered pattern may have any configuration that facilitates bone growth. Because the pattern is used to provide instructions for a computer-directed laser system, any pattern may be used and reproduced repeatedly to etch the pattern into the surfaces of a device. For example, complex geometrical patterns may be formed such as concentric rings or various fractal patterns. Additional options include autonomous generations of geometric progression where the relative subtleties of the continuity are reflected in the accent on historic topography and while appearing random are structural and learned progressions. Generation of such machine learning permits accenting the surfaces to be skewed, or retain kurtosis that accents bone healing.

The primary surface structures include recesses, which are voids or indentations extending from the outer surface but not necessarily through the device. The surfaces around the recess are referred to herein as prominences because the prominences protrude relative to the recesses. The recesses extend into a structurally solid feature of the implant device as directed by a non-random, engineered pattern. A prominence has a surface that appears relatively flat at a microlevel and serves as a reference for the depth of an adjacent recess.

With reference to FIG. 1B, each recess 140 has a mouth 142 that is defined by an adjacent prominence 150. Each recess 140 has at least one wall or a sidewall 144. The sidewall 144 of each recess 140 extends inwardly from the mouth 142 of the recess 140 to an end 148. FIG. 1B also depicts a mid-depth 146 of the sidewall halfway between the mouth 142 and the end 148. The same features are identified in FIG. 1C, which depicts a section of a device under greater magnification than that shown in FIG. 1A.

Each recess has a depth, length, and width. The depth of each recess, most, or at least a majority of the recesses as measured from its mouth to its end, is within a range of 100 nm to 2 mm, 500 nm to 2 mm, 1 micron to 2 mm, 10 microns to 1000 microns, 20 microns to 800 microns, 30 microns to 500 microns, and 40 microns to 200 microns. For example, the depth may be at least 200 microns, or at least 100 microns. The width of each recess, most, or at least a majority of the recesses is within a range of 1 micron to 2 mm, 60 to 500 microns, and 80 to 180 microns. The length of each recess is within a range of 10 microns to several millimeters. Additionally, the length aligns with the meta-morphology of cancellous bone which has trabecular width, mean trabecular volume, mean trabecular void, and inter-trabecular distances. The expectations of randomness is founded in data suggesting that tensile forces not only stabilize trabecular bone but enable and accentuate gene expression retaining the hallmark of bone-specific proteins, and that a void space is critical to the key assets of micro and nanoelasticity that are permissive to modeling and remodeling according to changing forces that are individual to each bone and to regions within each bone. When the length of the recess is much greater than the width of the recess then the recess is considered to be a trench or a trough depending on the configuration of the end. A recess with a sharp pointed end is a trench while a recess with a flat end is a trough. When the length and the width of a recess are about the same then the recess is considered to be a pit.

FIG. 1C depicts two recesses 140a and 140b that are relatively long. Recess 140a has an end 148a that is relatively flat such that it is shaped like a trough. In contrast, recess 140b has an end that is shaped like a trench because each opposing sidewall or wall ends abruptly at end 148b. However, both wall 144a and wall 144b are tapered inward along the depth of the recess such that their walls are inclined from the mouth to the end. The non-coplanar walls or arches flare outward from the end to the mouth of a recess. Another way of stating that the recesses flare outward going up or taper inward going down is to consider horizontal cross-sections measured along the depth of a recess from its mouth 142 to its mid-section 146 and further to its end 148, the horizontal cross-sectional shapes generally decrease in area for at least a majority of the recesses. Testing has shown that it is advantageous for the walls to have this increasing inclination from the surface and to extend inwardly because the inclined surfaces facilitate laser treatment and increased cross-sectional areas as derivatives of inclining laser-treated surfaces, as compared to those perpendicular to the surface prominence, and thereby provide for robust bone formation after implantation of a device. Some recesses in a set may differ from this configuration but the majority or most of the walls in a set have the tapered configuration. In some embodiments or in some sets of recesses, a recess may have walls that are essentially planar such that a wall is parallel to an opposing or adjacent wall and is perpendicular to the surface. For example, recesses formed by laser etching have walls that are mostly or all inclined walls while recesses formed by an additive process may have parallel walls, or inclined walls but lack the resolution of the femtosecond lasering process. Whether the walls are tapered inward from mouth to end or are essentially planar, these recesses are not undercut along their depths.

The computer-directed laser system as shown in FIG. 2 at 200 may be used to form the recesses 140. These recesses created by laser etching can be made either by moving the laser about the surface of the implant device 100; or the implant device 100 can be moved relative to the laser such that the recesses 140 are laid onto the exterior surfaces. Alternatively, the process may move both the implant and the laser simultaneously. The same principles apply to formation of the secondary surface structures 160. It has been found that it is advantageous to apply the laser at angles other than a 90° angle. For example, it is advantageous to apply the laser at up to 15° off of 90°. For example, these angles are helpful when forming the recesses. It is helpful to avoid refixturing the device during laser etching or laser treating the device such that the device only needs to be flipped over once. Limited refixturing helps to ensure that devices have identical patterns at the macrolevel while carrying forward the extent of evolving random nanosurfaces.

FIG. 1D and FIG. 1E are images of secondary surface structures 160 on a surface. The secondary surface structures 160 are also referred to herein as surface deformations, nano-sized features, and nanostructures. The surface deformations are modifications to a surface resulting from laser treating surfaces of a device including surfaces of the sidewalls of the recesses and surfaces around the mouth of each recess. The view in FIG. 1D is magnified and has a scale showing the length of 100 μm. FIG. 1E is a further magnified view of the surface with a scale showing the length of 1.00 μm. Both show surface deformations 160 in random configurations.

FIG. 1F is an image of a laser-treated surface under 200,000× magnification with a scale showing a length of 200 nm, so each tic is 20 nm. The surface was previously laser etched with the etched recesses having a maximum depth of about 100 um.

The surface deformations are especially random in comparison to the pattern of the recesses and are significantly smaller than the width of the recesses. The surface deformations include nano features that are discernable under magnification, such as 5,000× and 50,000× magnifications, and include nano-sized convex and concave structures that may be less than about 200 nanometers in width or diameter. These surface deformations may also have a height of less than about 200 nm. The surface deformations may be considered to be a comprehensive blasting, pitting, or surface peening of essentially the entirety of the surface at which the laser is directed. An area of the implant device that is laser treated has an increase of surface area after being laser treated. In some embodiments, the area that has been laser treated has a surface area that is about 100 times or more than the same size area of the surface of the structurally solid feature that is not laser treated to have surface deformations.

The surface deformations are only visible through powerful magnification which affords sub-micron resolution. As shown, these surface deformations exhibit very high surface areas in relation to their size. This large surface area creates advantageous regions to induce and to receive new bone growth. The bone-forming cells attach to these nano-sized surface deformations with greater ease and affinity than on solid untreated surfaces of the implant. The bone-forming cells become “activated” to form and remodel new bone through biologic changes in their morphology and biochemistry due to their interaction with this unique surface structure. Activation is furthered by cell-cell communication, fostering a tissue based organization that evolves from a cell-based induction.

The surface deformations may have dimensions of only a few nanometers (1 nm=10⁻⁹ m). The device may be modified to have larger surface deformations including those that are about, at least about, or no more than about 100, 150, 200, 250, 300, 350, 400, 450, 500, 550, 600, 650, 700, 750, 800, 850, 900, 950 nm, or more, or ranges including any two of the foregoing values. Most of the surface deformations are smaller than the width or depth of most of the recesses by an amount times about 5, 10, 50, 100, 150, 200, 250, 300, 350, 400, 450, 500, 550, 600, 650, 700, 750, 800, 850, 900, 950, 1000, 5000, 10000, or more, or ranges including any two of the foregoing values.

The surface deformations roughen the surface and provide structures for bone processes to become anchored. The bone processes function like multiple extensions or tentacles. Considering that a typical osteoblast has a diameter of about 25-50 microns and are morphologically diverse, the size and random configuration of the surface deformations is ideal for bone growth. The same principle applies to the short protrusions on the surface of the osteoblasts, processes, that are connected to adjacent cells and form a network structure.

The laser etching or texturing process used to form a pattern or random surface deformations varies depending on the device material and the structure to be created. For example, a laser beam having a power intensity wavelength in a range of about 470-570 nanometers may be used to form both the primary and the secondary surface structures with the exposure duration varied to be longer for the primary surface structures than for the secondary structures.

The laser etching may be performed by exposure to a “green laser,” which as used herein refers to a laser with the appropriate power required for industrial applications, with wavelengths varying between 470 to 570 nm, and more specifically between 515 nm to 532 nm. Many common green lasers are actually infrared lasers emitting natively 1064 nm light, but using a second crystal to double the frequency, and half the wavelength down to 532 nm thus providing green light. In such frequency-doubled IR lasers, the infrared light is then be filtered out to yield only green output. Lasers with higher wavelengths may also be used such as infrared lasers with a 1030 nm wavelength. However, a green laser is typically better because a green laser is relatively cooler, which creates less thermal damage such that the nanostructures are finer. Other lasers may also be utilized, for example, those using 300-nm wavelengths, those with parametric, tunable potential to achieve other wavelengths, and those with 0.15 nm or 10 kEv range.

The laser may be a “femtosecond laser,” which is a laser that emits ultrashort optical pulses with a duration well below 1 ps (10⁻¹² s), i.e., in a range of femtoseconds (1 fs=10⁻¹⁵ s). Femtosecond (FS) lasers thus belong to the category of ultrafast lasers or ultrashort pulse lasers (which also include picosecond lasers) having a pulse duration in the femtosecond range, or one quadrillionth of a second. The duration of exposure to a laser of such a FS laser impacts the depth of the structure that is created. Ultrashort (FS) pulse durations feature outstanding precision of machining and negligible rims or burrs surrounding the laser-irradiation zone. Consequently, additional mechanical or chemical post-processing steps are not required.

The depth of the recesses may be determined by the dwell time of the laser etching. A longer dwell time provides for deeper etching. The recesses may have identical or different depths. Those with different depths may have several sets of essentially identical depths. For example, a third of the recesses may have the same depth A, a second-third portion have the same depth B, and the third portion have the depth C. By allowing for varied depths, the recesses may provide differing bone growth such that the variety improves the overall success.

As indicated above, the implant can be made of a metal material. When the metal material is exposed to the laser, the material exhibits an increased oxidation at the surface and chemically alters the material to enhance osteoinductivity for new bone growth formation when implanted. Thus, there is a metallurgical change in material, and the metal is no longer chemically pure. For example, the metal may be a titanium alloy and when exposed to a laser, the surface is enhanced with an oxide. An example of a preferred titanium alloy comprises 90 percent titanium, 6 percent aluminum and 4 percent vanadium. When such a titanium alloy is exposed to a laser then the laser alters the chemical structure at the surface by forming an oxide, which may be titanium oxide, aluminum oxide, or vanadium oxide. The oxide enhances the new bone growth features at the surface configured to mimic trabecular bone, and to vary elemental valence and order charge variation across the surface. Laser exposure may also introduce electrical conductivity such that there is an electrical charge at the surface. Such a surface makes it easier for bone processes to attach and become anchored to the surface to facilitate bone growth.

As also indicated above, the implant can also incorporate ceramics as part of its composition. A cermet can combine attractive properties of both a ceramic, such as high temperature resistance and hardness, and stiffen features of a metal, such as the ability to undergo plastic deformation. Depending on the physical structure of the material, cermets can also be considered as metal matrix composites, but cermets are usually less than 20% metal by volume. Exemplary cermets include Zirconia-titanium sintered constructs. Cermets have been used in the manufacture of resistors (especially potentiometers), capacitors, and other electronic components. In the context of biologic systems, which are based on membrane polarization and nano-voltage, it is not surprising that Zirconia-titanium sintered constructs have been known for some time to enhance bone cell response.

Examples of Structures Formed Via Subtractive Process

Following are examples of osteoinductive surfaces that may be used to manufacture devices with osteoinductive surfaces via subtractive (non-additive) processes. The exemplary configurations and conditions are given by way of example, and not be limitation.

### Example 1

A test disk such as test disk 110 in FIG. 4A was analyzed. The test disk had a diameter of 9 mm, an unetched border that is 0.5 mm wide, and primary surface structures, which comprises an etched area having a diameter of 8 mm. FIG. 4B depicts the test disk in a plan view with a cutting line 4C-4C taken from two points identified as A and B. The length of the line between points A and B is 11 mm. The depth of the recesses and surfaces along the line is depicted in a graph in FIG. 4C. The recesses with greatest depths have a recess of about 0.18 mm and those with that are the most shallow have a depth of about 0.02 mm. Other recesses have a depth of about 0.04 mm and 0.12 mm. There are 18 different recesses along the line between points A and B but only four different depths. Of course, infinite widths may be associated with the differences in depth along the surface relative to different lines drawn either perpendicular or skewed to the conformation. For example, much like the topography of the Grand Canyon in North America might vary depending on the trajectory between the north and south elevations, that same variation applies depending upon the site line connecting the radial potential of a point to any other point on the opposite face of the trench.

An enlarged section of the test disk is shown in FIG. 4D with two points identified as C and D and a cutting line 4E-4E connecting the two points with points 1-12 between points C and D. The length of the line between points C and D is 7500 μm (7.5 mm). FIG. 4E depicts the depths of the recesses and surfaces at points 1-12 as measured by the profilometer. The depths of the recesses and surfaces at points 1-12 are summarized in the table below.

Recesses 1-12 in FIG. 4E

| Recess | Depth | |
|---|---|---|
| #1 | 143.81 | μm |
| #2 | 169.64 | μm |
| #3 | 40.19 | μm |
| #4 | 43.17 | μm |
| #5 | 113.45 | μm |
| #6 | 193.81 | μm |
| #7 | 174.99 | μm |
| #8 | 183.62 | μm |
| #9 | 91.04 | μm |
| #10 | 99.73 | μm |
| #11 | 128.35 | μm |
| #12 | 41.44 | μm |

FIG. 4F depicts the same enlarged section depicted in FIG. 4D and a few select recesses identified as recesses #1-#6. Recesses #1-#4 are examples of pits. Recesses #5-#6 are long trenches. The table below lists the average depth, the maximum depth, and the perimeter of each recess.

Recesses 1-6 in FIG. 4F

| Recess | Avg. Depth | Max. Depth | Perimeter |
|---|---|---|---|
| #1 | 160.15 μm | 195.43 μm | 801 μm |
| #2 | 161.82 μm | 195.26 μm | 779 μm |
| #3 | 172.61 μm | 209.98 μm | 762 μm |
| #4 | 167.29 μm | 207.47 μm | 779 μm |
| #5 | 162.57 μm | 212.11 μm | 12073 μm |
| #6 | 163.02 μm | 208.55 μm | 12143 μm |

The average depth has a maximum of 160.15 μm, a minimum of 172.61 μm, an overall average of 164.58 μm, standard deviation of 4.19 μm, and 3 Sigma of 12.58 μm. The maximum depth has a maximum of 195.26 μm, a minimum of 212.11 μm, an overall average of 204.80 μm, standard deviation of 6.83 μm, and 3 Sigma of 20.50 μm.

FIG. 4F also features horizontal line EF and vertical line GH taken respectively between points E and F and between points G and H. The depths of these lines are also depicted in FIG. 4G and FIG. 4H.

### Example 2

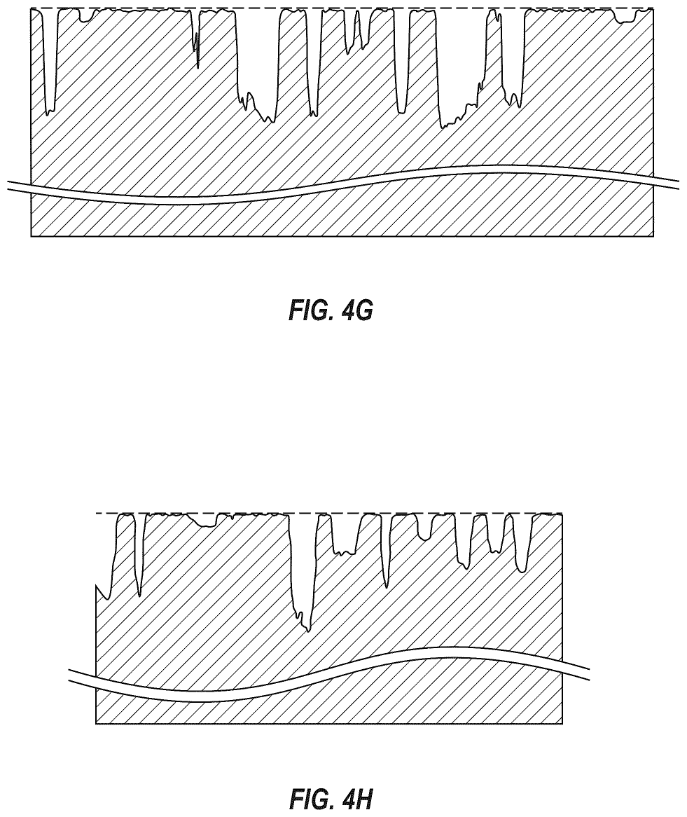

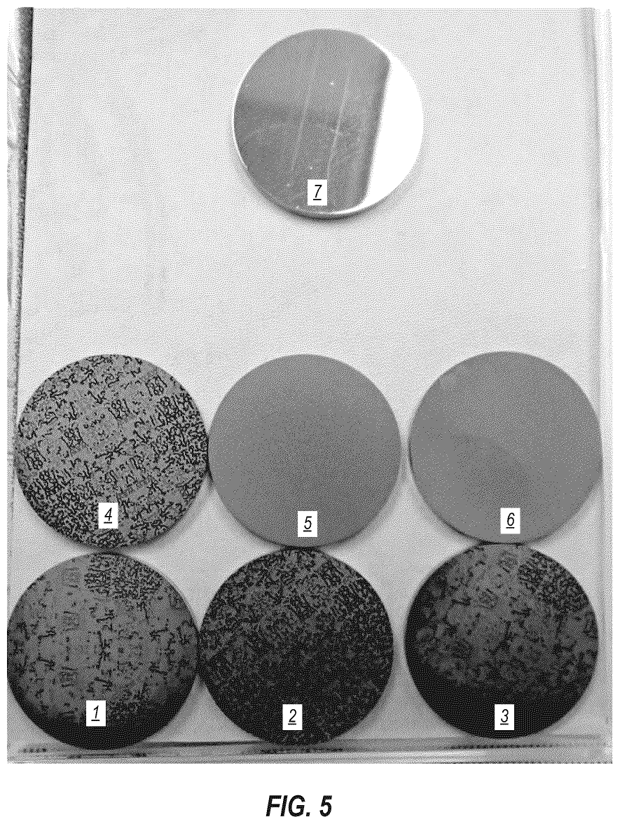

This example provides comparative information for different configurations on several disks as shown in FIG. 5, which depicts seven disks that were samples subjected to comparative analysis when viewed via scanning electron microscopy (SEM) in which the samples have differing recess trench depths via laser etching, use of surface texturing, and a control. The samples were formed from medical grade titanium alloy, sometimes referred to as T5, but generally formed as an empiric amalgam of 90% titanium, 4% aluminum, and 6% vanadium. Sample #1 and sample #3 were disks that were etched to form recesses having a depth of 100 microns. Sample #2 and sample #4 were etched more deeply to have recesses with a depth of 200 microns. Samples #1-#4 were also surface treated with a laser that blasted the surfaces of the disk to yield nano-textured surfaces with nano-sized features. The laser etching and laser treating for Samples #1-#4 were performed under atmosphere. Sample #5 and sample #6 were not laser etched to form macro-sized or surface recesses and were only treated with a laser that blasted the surfaces of the disk to yield nano-textured surfaces with nano-sized features. Sample #7 was the control as it was polished but was not laser etched or laser blasted to yield nano-sized feature. Images of the disks were taken with varying levels of magnification on a 3D digital microscope made by Hirox.

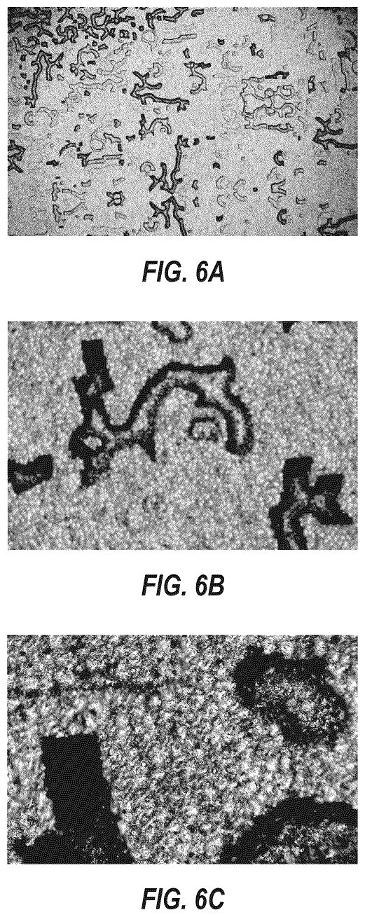

Sample #1 is shown in FIGS. 6A-6C. The deepest recesses in Sample #1 are shown in the darkest shade and have a depth of 100 microns. FIG. 6A shows a section of Sample #1 having a length of 2000 μm at 20× magnification. FIG. 6B shows a section of Sample #1 in FIG. 6A with a length of 500 μm and further magnified to a level of 120× magnification. FIG. 6C shows a section of Sample #1 in FIG. 6A with a length of 500 μm and further magnified to a level of 200× magnification.

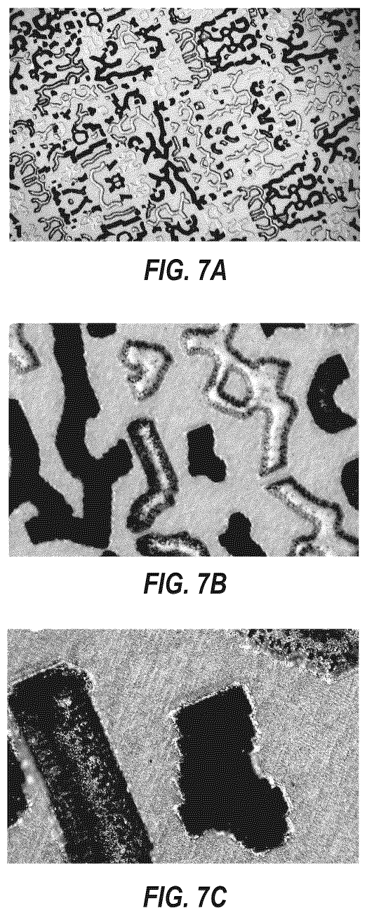

Sample #2 is shown in FIGS. 7A-7C. The deepest recesses in Sample #1 are shown in the darkest shade and have a depth of 200 microns. FIG. 7A shows a section of Sample #2 having a length of 2000 μm at 20× magnification. FIG. 7B shows a section of Sample #2 in FIG. 7A with a length of 500 μm and further magnified to a level of 120× magnification. FIG. 7C shows a section of Sample #2 in FIG. 7A with a length of 500 μm and further magnified to a level of 200× magnification. As is evident by comparing FIGS. 7A-7C with FIGS. 6A-6C, the recesses are darker in FIGS. 7A-7C since they are deeper.

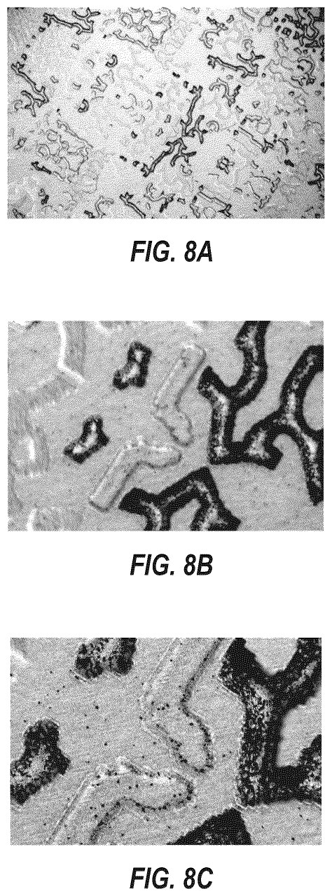

Sample #3 is shown in FIGS. 8A-8C. The deepest recesses in Sample #3 are shown in the darkest shade and have a depth of 100 microns. FIG. 8A shows a section of Sample #3 having a length of 2000 μm at 20× magnification. FIG. 8B shows a section of Sample #3 in FIG. 8A with a length of 500 μm and further magnified to a level of 120× magnification. FIG. 8C shows a section of Sample #3 in FIG. 8A with a length of 500 μm and further magnified to a level of 200× magnification. Sample #3 repeats the pattern from Sample #1 with greater recess depth so as expected, they appear the same with respect to their trench pattern but the trenches in Sample #3 are more darkly depicted. However, the surface treatment differs as shown best by comparing FIGS. 6B and 6C with FIGS. 8A and 8C. Since the surface treatment is random, this is the expected outcome.

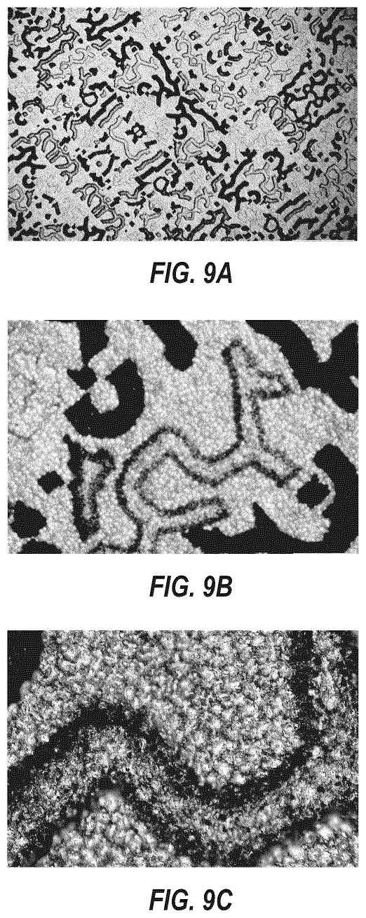

Sample #4 is shown in FIGS. 9A-9C. The deepest recesses in Sample #4 are shown in the darkest shade and have a depth of 200 microns. FIG. 9A shows a section of Sample #4 having a length of 2000 μm at 20× magnification. FIG. 9B shows a section of Sample #4 in FIG. 9A with a length of 500 μm and further magnified to a level of 120× magnification. FIG. 9C shows a section of Sample #4 in FIG. 9A with a length of 500 μm and further magnified to a level of 200× magnification. Sample #4 repeats the pattern from Sample #2 with greater recess depth so as expected, they appear the same with respect to their trench pattern but the trenches in Sample #2 are more darkly depicted. However, the surface treatment differs as shown best by comparing FIGS. 7B and 7C with FIGS. 9A and 9C. Since the surface treatment is random, this is the expected outcome.

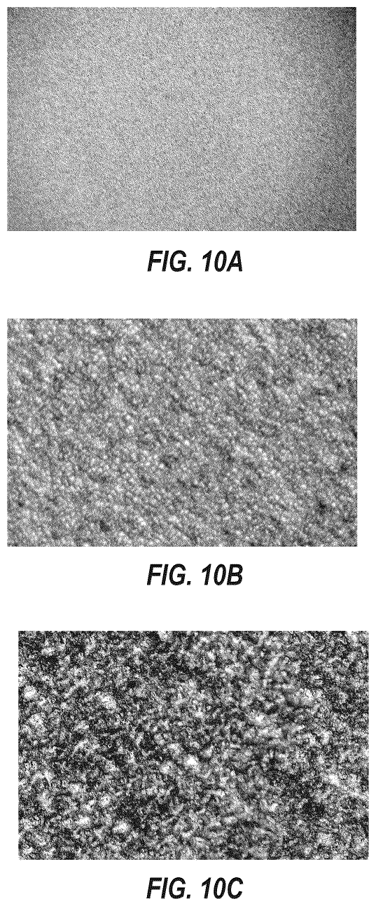

Sample #5 is shown in FIGS. 10A-10C. Sample #5 was treated with a laser that blasted the surfaces of the disk to yield nano-textured surfaces with nano-sized features, which are also referred to as surface modifications or secondary surface structures or features. FIG. is a section of the disk, at a level of 20× magnification of a section of Sample #5 having a length of 2000 μm. FIG. 10B is a SEM image of a section of Sample #5 in FIG. 10A with a length of 500 μm and further magnified to a level of 120× magnification. FIG. 10C is a SEM image of a section of Sample #5 in FIG. 10A with a length of 200 μm and further magnified to a level of 200× magnification.

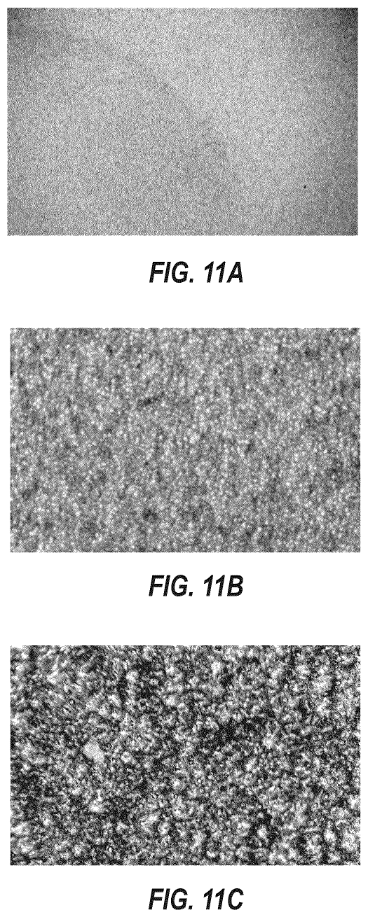

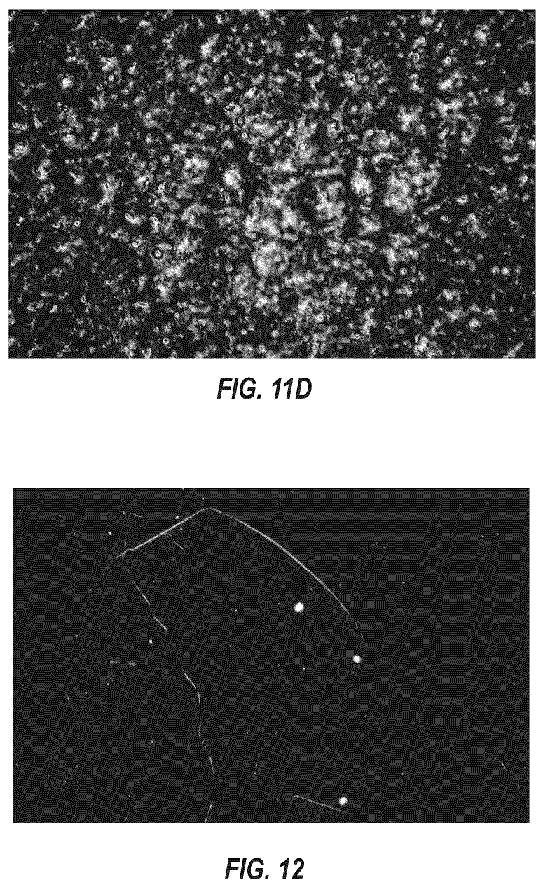

Sample #6 is shown in FIGS. 11A-11D. Like Sample #5, Sample #6 was treated with a laser that blasted the surfaces of the disk to yield nano-textured surfaces with nano-sized features. FIG. 11A is a section of the disk, at a level of 20× magnification of a section of Sample #6 having a length of 2000 μm. FIG. 11B is a SEM image of a section of Sample #6 in FIG. 11A with a length of 500 μm and further magnified to a level of 120× magnification. FIG. 11C is a SEM image of a section of Sample #6 in FIG. 11A with a length of 200 μm and further magnified to a level of 200× magnification. As expected, the images of FIGS. 11A-11C look similar to the images of FIGS. 10A-10C. However, a pattern cannot be discerned when comparing the samples as the same levels of magnification because the laser treatment is random. The texture of the concave and convex structures are best seen in FIG. 11D, which is a SEM image of a section of Sample #6 in FIG. 11A with a length of 200 μm and further magnified to a level of 400× magnification.

Sample 7 is a control disk and is shown in FIG. 12. FIG. 12 is a SEM image of a section of Sample #7 with a length of 500 μm and magnified to a level of 120× magnification. As expected, its surface topography is essentially flat, very smooth and featureless—not roughened like the samples having a randomly roughened topography due to laser treatment.

### Example 3

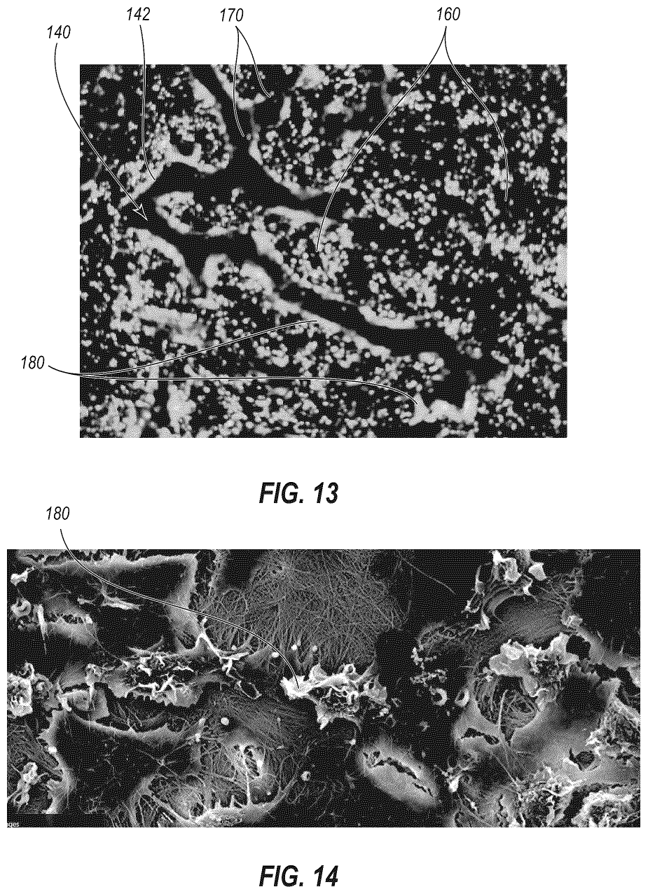

FIG. 13 is a composite image of photographs that were taken when focused on a surface that was laser etched to form trenches 140 and that has laser-treated surfaces 160. FIG. 13 identifies a few nonviable cells 170 and numerous healthy bone cells 180. The healthy bone cells 180 are shown growing around trenches 170, particularly on the mouths 142 of the trenches 140. Healthy bone cells 180 are not shown growing in the trenches 140 only because the photographs were focused on the surface and were out of focus in the trenches 140 due to the depths of the trenches 140. Viability was assayed separately from expression of genes known to define osteoblastic lineage. By using transgenic insertions responsive to fluorescent light at prescribed wavelengths, it was possible to interpret that cells on the surface determined to be viable were in register with markers identifying as bone cells.

FIG. 14 is an SEM image showing the bone growth, in vitro, on a laser treated device. Healthy bone cells 180 are shown robustly growing by the portions that are white, which include numerous bone processes or extensions. Testing also showed proliferation, gene expression, and robust production of bone specific proteins bone sialoprotein and dental matrix protein. The image provided in FIG. 14 was taken after just 14 days after cell exposure and shows robust cell attachment and bone growth. Such a rate of bone formation is unprecedented and indicates that healthy in vivo regeneration should be expected after implantation. More specifically, this testing indicates that the surfaces that have been modified as disclosed herein provide for rapid bone growth and are likely to hasten and improve fusion rates of bone to implant surface. Thus, it may be possible to achieve these objectives without bone graft or bone graft substitute products.

### Example 4

After etching and blasting surfaces of a disk formed from a titanium alloy comprising 90 percent titanium, 6 percent aluminum and 4 percent vanadium, there was an apparent variation in oxidation observed in the electron dispersive spectroscopy (EDS). The oxidation may result in titanium oxide, aluminum oxide, or vanadium oxide.

Vanadium adds stiffness to titanium and has a bonding pattern similar to phosphorous which has been suggested to be a trait that accentuates a role as an insulin mimetic and positively affects bone cell proliferation. As a central tenet of the surface modification, incorporating positive effects on osteoblastogenesis is an essential part of the bone healing process. Insulin has been shown to improve bone healing in both normal and diabetic bone healing models, and insulin mimetic compounds such as zinc chloride (ZnCl₂) and vanadyl acetylacetonate (VAC) have also been shown to improve bone healing. Vanadium might operate by similar methods to zinc.

### Example 5

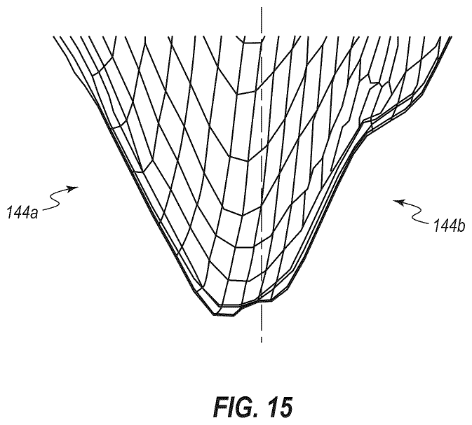

FIG. 15 is a schematic representation of a recess formed by laser etching a solid feature of an implant device. The recess has an end that is relatively pointed and the width and length are about the same such that the recess is shaped like a pit. The sidewalls are tapered inward along the depth of the recess or stated otherwise flare outward from the end outward going up or taper inward going down.

The shape of the recess was based on the x, y, and z axes derived from trabecular structure, thus the shape of the pit was inherently asymmetric. For example, bisection of the pit as shown in FIG. 15 into two portions, 144a and 144b, shows some symmetric areas and some asymmetric areas. The recess is essentially a pit but is not perfectly conical. Thus, the use of trabecular bone as a model does introduce some asymmetry.

Structures Formed Via Primarily Additive Process

According to one embodiment, a method of making a device, such as a spinal implant fusion device, via an additive process comprises the steps of: fabricating a n implant body structure using 3D printing to create the implant body structure; additively building the body structure having a superior load bearing surface and an inferior load bearing surface and a wall structure; and wherein the body structure has at least a portion of the body structure having a plurality of interconnected struts forming porous walls with openings or passages extending inwardly from an exterior surface to a depth of 1.0 mm or greater forming a porous portion with a void volume to solid mass volume mimicking trabecular bone. Alternatively, the 3D printed structure may be completely or substantially solid with a surface structure comprised of the interconnected arcs that are raised, or created like trenches or troughs that appear to be cut into the surface but were created through 3D printing.

The average or nominal ratio of void volume to mass volume in the porous portion may varying depending on the objective such as replicating the trabecular bone in an adult male or female. For example, the density may be in a range of 65 percent or more such as 75 percent. A density of about 75 percent replicates that of trabecular bone in an adult male.

The struts of the porous walls are curved or arch shaped with openings communicating with adjacent walls. The porous portion of the implant body structure extends at least partially across the implant body structure exterior surfaces forming conduits for fluid passage throughout the device. The curved or arch shaped struts of the walls create a load bearing capacity to withstand vertical loads without collapsing. The implant fusion device has the superior load bearing surface and the inferior load bearing surface having nano channels etched on exposed surfaces. The etching is created through a subtractive laser process.

In another embodiment, a method of making a device such as a spinal implant fusion device comprises the steps of: providing a 3D printed implant body structure; and subsequent subtractive laser etching which results in nanometer-level structure on at least a portion of a surface or surfaces of the implant body structure, the nanometer structure creating new bone growth attachment features to enhance osteoinductivity of the spinal implant or orthopedic fusion device. The 3D printed structure may be solid or relatively solid prior to use of the laser etched subtractive process that results in a nanotechnology level of surface for the induction of bone formation and growth.

The laser etched nanometer structural features are made into a network of features in either a random pattern or an organized pattern. The laser etching is formed by emitting laser beams unobstructed to the surfaces of the implant. The method of making a device such as a spinal implant fusion device or other orthopedic or bone implant further has the step of moving a laser about the implant body structure to create the network of features or the method has the step of moving the implant body structure about a laser to create the network of features.

In an additional embodiment, a combination of 3D printing and laser etching to manufacture a device such as a spinal implant fusion device or orthopedic or bone device comprises the steps of: fabricating an implant body structure using 3D printing to create the implant body structure; additively building the body structure having a superior load bearing surface and an inferior load bearing surface and a wall structure; wherein the body structure has at least a portion of the body structure having a plurality of interconnected struts forming porous walls with openings extending inwardly from an exterior surface to a depth of 1.0 mm or greater to form a porous portion with a void volume to solid mass volume mimicking trabecular bone; and laser etching to yield nano-sized features on at least a portion of the exterior surface or surfaces of the implant body structure, the nano-sized features such as channels creating new bone growth attachment features to enhance osteoinductivity of the spinal implant fusion device.

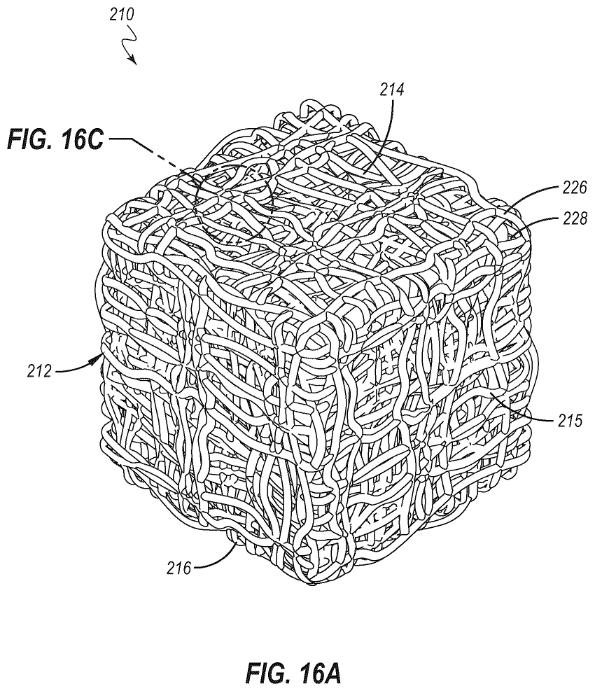

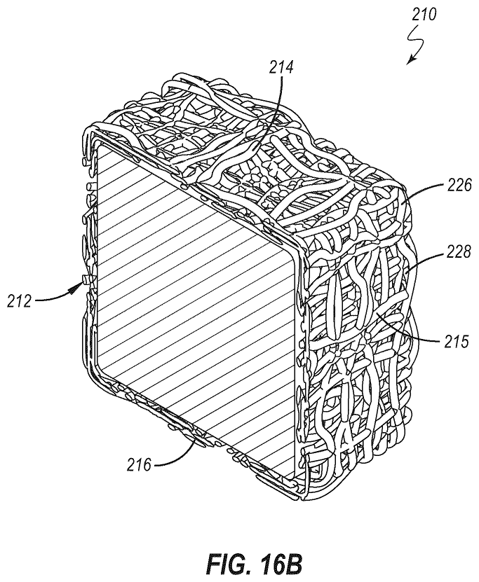

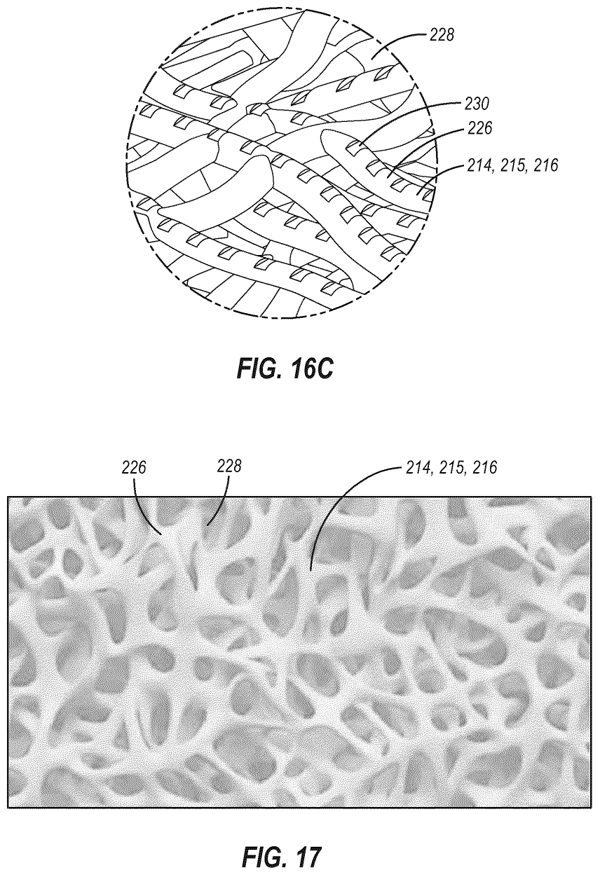

FIG. 16A is an exemplary embodiment of a device 210 such as a spinal implant fusion device formed at least in part by an additive process. The configuration of the implant device 210 as illustrated has a first or superior surface 214, a second or inferior surface 216 and side surfaces 215 that surround and form the exterior surfaces of the implant body structure 212. When used as a spinal implant fusion device, the first surface 214 and second surface 216 provide the device 210 with surfaces that upon implantation between two adjacent vertebral bodies will support the bone structure of the adjacent vertebral bodies. These first and second surfaces 212, 216 may be direct contact with the bone structure of the adjacent vertebral bodies of the patient upon implantation of the device 10 for a procedure where an implant fusion device is being implanted to correct a degenerative condition or other condition in a patient. As shown, the exemplary embodiment is merely an example of a simple shape. In addition to the cube shape depicted in FIG. 16A, any number of shapes can be used and can be any number of polygonal shapes of various shapes and sizes. For example, the device may be rectangular, oblong or elongated. A cylindrical device with a circular side can be used. Similarly, the device can have a pentagonal or hexagonal shape where the sides are not circular. When designed for use as a spinal implant fusion device, the only limitation for the shape is having a size that is sufficient to support the load between the adjacent vertebral bodies to function as a proper implant fusion device.

With reference to FIG. 16B, a cross-sectional view is taken from FIG. 16A along cutting line 16B-16B of FIG. 16A. The cross-sectional view shows the interior structure of the device 210. As shown, the body structure 212 of the device 210 may have a solid central area or region and an external region with ratio of void volume to mass volume that is relatively high and replicates trabecular bone, more particularly cancellous trabecular bone wherein the high porosity creates open pathways for fluid to move in and out similar to what happens in natural bone. The body structure 212 of the implant device 210 is formed by 3D or additive printing yielding exterior portions made with a plurality of interconnected struts 226. The struts 226 are curved or arched and spaced between connections with openings 228 forming a porous wall having a porosity that replicates that of an adult male or female depending on the implant being produced.

The region having a high ratio of porosity extends inward towards a central region of the device 210. As the device extends from the perimeter to the exterior surfaces 214, 215, 216 of the device 210 this ratio of void volume to mass volume can be reduced dramatically, this occurs as the 3D building of the device is being performed. As such, the exterior surfaces 214, 215, 216 the porous walls may extend approximately 1 mm or greater into the interior from the exterior surface with a center portion of the body structure having a much reduced ratio of void volume to mass volume. This reduced ratio is more tightly compacted creating a core inside the device with a porous structure around the entire device 210. This enhances the structural strength of the device 210 and provides a superior bone generating exterior surface or surfaces of the more open porosity with the interior core of the body structure 212 providing high strength.

Optionally, the porous structure of interconnected struts 226 can be made to extend throughout the implant body structure if so desired. In practice, it has been found that the depth of the surfaces mimicking the trabecular bone of at least 1 mm in depth is ideal for new bone formation and therefore the 3D manufacturing of the implant may be made simpler and less expensively by limiting the depth to 1 mm or greater. Additionally, the superior 214 and inferior 216 surfaces should have the porous trabecular features, but the side walls may be solid as an optional way to manufacture the device.

With reference to FIGS. 16C and 17, a portion of the exterior surface 214, 215, 216 is shown. This porous exterior surface can be along the surface of the first surface 214, second surface 216 or side surface 215 or all of these surfaces.

As shown in FIGS. 16C and 19A-19B, nanostructures such as nano channels 230 may be formed via a laser etching machine such as that depicted by the laser system at 200 in FIG. 2. These nano channels 230 can be laid in a network 218 either in an organized uniform pattern or a random non-uniform pattern throughout the exterior surfaces 214, 215, 216. Ideally, these nano channels 230 are created at least along the first and second surfaces 214, 216 of the implant device 10. The nano channels 30 are small laser etched cuts that can be laid out along the entire exterior surfaces in a subtractive laser etching process. These nano channels 230 created by laser etching can be made either by moving the laser 200 about the exterior surface 214, 215, 216 of the implant device to form the nano channels 30 as shown in FIG. 19A; or the device 210 may be moved relative to the laser such that the nano channels 230 are laid onto the exterior surfaces 214, 215, 216 as shown in FIG. 19B. The nano channel features individually create an improved osteoinductive effect at the surface of the implant device 210. This means that the formation of new bone once implanted into the patient can be accelerated and the network 218 of nano channels 230 provide features that help assist in providing attachment locations for the new bone formation. This is an important feature that is provided in the current invention and is ideal in that it does not require smooth or flat exterior surfaces to form the channels which are effectively etched or burned into the exterior surface. The channels can be created as long as the path of the laser beam is unobstructed. As a result, even though the porous walls of exterior surfaces have slight undulations 220, 222 and openings, the network 218 of nano-channels 230 can be formed regardless of this and not limited to the topography of exterior surfaces commonly found in implant devices that are molded or otherwise have smooth exterior surfaces. In fact, the nano channels 230 can be found formed at varying depths where the openings allow the laser beam to pass. The nano channels preferably have a width and a depth of 10 nano meters or greater up to 1000 nano meters. These features are very small and unlike micro channel laser etching, the nano channels can be etched extremely quickly due to their small size.

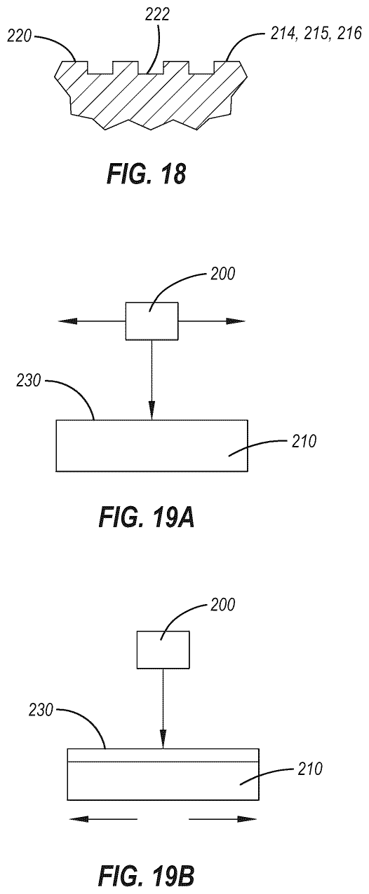

FIG. 18 is a simplified schematic outline of a portion of an exterior surface 214, 215, 216 of an alternative embodiment. The exterior surface 214, 215, 216 has an undulating feature such that the exterior surface has protrusions 220 projecting outwardly slightly and channels or troughs 222 that are slightly recessed. These features create an undulating surface and when formed on an implant fusion device, they enhance the ability of the device 210 to create space between the adjacent vertebrae after the device 10 is implanted. Additionally, this entire surface is then treated using laser etching to create nano channels 230 best shown in FIG. 16C.

The embodiments described above detail methods of making a device such as an orthopedic device with a surface pattern mimicking trabecular bone. The body of the device may be made by any conventional process or by 3D printing. The surface may be formed by either a subtractive or an additive process. The surface may be treated to form nanostructures whether the body is formed by a subtractive or an additive process. Such surface structures are nanometer in scale and are biologically active in inducing bone growth.

Although the structures, devices, methods, and systems have been described in accordance with particular embodiments, one of ordinary skill in the art will readily recognize that many variations to the particular embodiments are possible, and any variations should therefore be considered to be within the spirit and scope disclosed herein. Accordingly, many modifications may be made by one of ordinary skill in the art without departing from the spirit and scope of the appended claims.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited.

Any range disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any of the claims beginning with claim [x] and ending with the claim that immediately precedes this one,” where the bracketed term “[x]” is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claim 1, 2, or 3, with these separate dependencies yielding three distinct embodiments; claim 5 can depend from any one of claim 1, 2, 3, or 4, with these separate dependencies yielding four distinct embodiments; and so on.

Recitation in the claims of the term “first” with respect to a feature or element does not necessarily imply the existence of a second or additional such feature or element. Elements specifically recited in means-plus-function format, if any, are intended to be construed to cover the corresponding structure, material, or acts described herein and equivalents thereof in accordance with 35 U.S.C. § 112 ¶6. Embodiments of the invention in which an exclusive property or privilege is claimed are defined as follows.

1. A method of manufacturing an implant device configured to have at least one osteoinductive feature to enhance new bone formation after implantation, the method comprising:
providing an implant device;
laser etching one or more surfaces of the implant device to create a plurality of recesses extending into the implant device;
wherein each recess has a mouth, an end opposite from the mouth, and a sidewall extending between the mouth and the end and into the implant device;
wherein the recesses are shaped as directed by a non-random, engineered pattern; and
wherein the mouth of each recess has a width, and most of the mouths have a width within a range of about 100 nm to about 2 mm; and
laser treating surfaces of the implant device including surfaces of the sidewalls of the recesses and surfaces around the mouth of each recess to create surface deformations that are random in comparison to the pattern of the recesses and are significantly smaller than the width of the recesses.

2. The method of claim 1, wherein each recess has a depth as measured from its mouth to its end, and most of the recesses have a depth within a range of about 1 micron to about 2 mm.

3. The method of claim 1, wherein most of the surface deformations have a length or width of less than about 200 nanometers.

4. The method of claim 1, wherein an area of the implant device that is laser treated has an increase of surface area after being laser treated.

5. The method of claim 1, wherein the one or more surfaces of the implant device are chemically altered by oxidation due to laser etching or laser treating the one or more surfaces.

6. The method of claim 1, wherein the implant device is a titanium alloy, and wherein titanium oxide is formed at the surface by the laser etching.

7. The method of claim 1, wherein the sidewall of each recess has a mid-depth halfway between the mouth and the end, and wherein at least a majority of the recesses are structured such that the recess progressively narrows because the sidewall is inclined from its mouth to its end or at least to its mid-depth.

8. The method of claim 1, wherein most of the surface deformations are at least 5 times smaller than the width of most of the recesses.

9. A method of manufacturing an implant device configured to have at least one osteoinductive feature to enhance new bone formation after implantation, the method comprising:
providing an implant device with one or more surfaces that have a plurality of recesses extending into the implant device;
wherein each recess has a mouth, an end opposite from the mouth, and a sidewall extending between the mouth and the end and into the implant device;
wherein the recesses are arranged in a non-random, engineered pattern; and
wherein each recess has a depth, and most of the recesses have a width within a range of about 1 micron to about 2 mm; and
laser treating surfaces of the implant device including surfaces of the sidewalls of the recesses and surfaces around the mouth of each recess to create surface deformations that are random in comparison to the pattern of the recesses and are significantly smaller than the width of the recesses;
wherein most of the surface deformations, as seen under high magnification, have a length or width of less than 200 nanometers.

10. The method of claim 8, wherein the recesses are formed by laser treating the one or more surfaces of the implant device.

11. The method of claim 9, wherein an area of the implant device that is laser treated has an increase of surface area after being laser treated.

12. The method of claim 9, wherein the one or more surfaces of the implant device are chemically altered by oxidation due laser etching or laser treating the one or more surfaces.

13. The method of claim 9, wherein the implant device is a titanium alloy, and wherein titanium oxide is formed at the surface by the laser etching.

14. An implant device configured having at least one osteoinductive feature to enhance new bone formation after implantation, the implant device comprising:
one or more surfaces of the implant device having a plurality of recesses extending into the implant device;
wherein each recess has a mouth, an end opposite from the mouth, and a sidewall extending between the mouth and the end;
wherein the sidewall extends inwardly from the mouth to the end and has a mid-depth halfway between the mouth and the end;
wherein each recess has a depth as measured from its mouth to its end, and at least a majority of the recesses have a depth within a range from about 10 to about 500 microns; and
wherein at least a majority of the recesses are structured to narrow progressively inward because the sidewall of the recess that is structured to narrow progressively inward, is inclined from its mouth to its end or at least to its mid-depth.

15. The implant device of claim 14, wherein the implant is made of a metal.

16. The implant device of claim 15, wherein the metal is a titanium alloy.

17. The implant device of claim 16, wherein the titanium alloy is 90 percent titanium, 6 percent aluminum and 4 percent vanadium.

18. The implant device of claim 16, wherein the implant device has at least one surface that comprises titanium oxide.

19. The implant device of claim 14, wherein the recesses are arranged in a non-random, engineered pattern; and
wherein the one or more surfaces of the implant device comprise surface deformations that are random in comparison to the pattern of the recesses and are significantly smaller than the depth of the recesses.

20. The implant device of claim 14, wherein the recesses have varying depths such that not all of the recesses have essentially the same depth.
